(19) United States
(12) Patent Application Publication
(10) Pub. No.: US 2020/0046002 A1
(43) Pub. Date: Feb. 13, 2020

(54) METHOD FOR PREPARING LIPID-RICH COMPOSITIONS OF MICROALGA FLOUR WITH OPTIMIZED ORGANOLEPTIC PROPERTIES

(71) Applicant: Corbion Biotech, Inc., South San Francisco, CA (US)

(72) Inventors: Amandine Druon, Lille (FR); Marie Le Ruyet, Lille (FR); Laurent Segueilha, Marquette Lez Lille (FR)

(73) Assignee: Corbion Biotech, Inc., South San Francisco, CA (US)

(21) Appl. No.: 16/540,748

(22) Filed: Aug. 14, 2019

Related U.S. Application Data
(63) Continuation of application No. 14/911,743, filed on Feb. 12, 2016, now abandoned.

(30) Foreign Application Priority Data
Aug. 13, 2013 (FR) 1357996

(51) Int. Cl. A23L 17/60; A23L 2/52; C12N 1/12

(52) U.S. Cl. CPC A23L 17/60; A23V 2002/00; C12N 1/12; A23L 2/52

(57) ABSTRACT

The present invention relates to a method for preparing, by fermentation, lipid-rich compositions of microalga flour having optimised organoleptic properties, characterised in that the microalgae are cultured in a minimal culture medium.

The present invention relates to a novel process for the preparation, by the fermentative route, of compositions of flour of lipid-rich microalgae of the Chlorella genus, exhibiting an optimized sensory profile, which makes it possible to incorporate them in food formulations without generating undesirable aromas.

### PRESENTATION OF THE STATE OF THE ART

Historically requiring “only water and sunlight” to grow, algae have for a long time been considered to be a source of food.

There exist several species of algae which can be used in food, the majority being “macroalgae”, such as kelp, sea lettuce (Ulva lactuca) and red algae of Porphyra (cultivated in Japan) or dulse (Palmaria palmata) type.

However, in addition to these macroalgae, there are also other sources of algae represented by the “microalgae”, that is to say photosynthetic or nonphotosynthetic unicellular microscopic algae, of or not of marine origin, cultured for their applications in biofuels or food.

For example, spirulina (Arthrospira platensis) is cultured in open lagoons (under phototropic conditions) for use as food supplement or incorporated in small amounts into confectionery or drinks (generally less than 0.5% weight/weight).

Other lipid-rich microalgae, including certain species of Chlorella type, are also very popular in Asian countries as food supplements (mention is made of microalgae of the genus Crypthecodinium or Schizochytrium which produce omega-3 fatty acids).

The production and the use of the flour of microalgae of Chlorella type are, for example, described in the documents WO 2010/120923 and WO 2010/045368.

The oil fraction of the flour of microalgae, which can be composed essentially of monounsaturated oils, can offer nutritional and health advantages in comparison with the saturated, hydrogenated and polyunsaturated oils often found in conventional foodstuffs.

When it is desired to industrially manufacture microalgal flour powders from their biomass, major difficulties remain, not only from the technological viewpoint but also from the viewpoint of the sensory profile of the compositions produced.

This is because, while algal powders, for example manufactured with algae photosynthetically cultured in open-air ponds or by photobioreactors, are available commercially, they have a dark green color (associated with chlorophyll) and a strong unpleasant taste.

Even formulated in foodstuffs or as nutritional supplements, these algal powders still impart this green color, which is visually not very attractive, to the foodstuff or to the nutritional supplement and have an unpleasant fishy taste or the savor of marine algae.

Furthermore, it is known that certain species of blue-green algae naturally produce odorous chemical molecules, such as geosmin (trans-1,10-dimethyl-trans-9-decalol) or MIB (2-methylisoborneol), generating earthy or moldy odors.

With regard to the Chlorellae microalgae, the descriptor commonly accepted in this field is the “green tea” taste, somewhat similar to other green plant powders, such as green barley powder or green wheat powder, which taste is attributed to its high chlorophyll content.

Their savor is normally only masked when they are mixed with highly savored vegetables or citrus fruit juices.

There thus still exists an unsatisfied need to have available compositions of flour of microalgae of the Chlorella genus of suitable organoleptic quality making possible the use of these compositions in a greater number of and more diversified foodstuffs.

### SUMMARY OF THE INVENTION

The applicant company has found that it is possible to meet this need by providing a novel process for the preparation, by the fermentative route, of compositions of flour of lipid-rich microalgae of the genus Chlorella.

Thus, the present invention relates to a fermentation process in which the microalgae are cultured in a “minimum” culture medium.

Preferably, the minimum medium:
contains a reduced amount of source of sulfur and/or
is deprived of one or more vitamins of the B group, in particular of vitamins of choline and inositol type.

According to one embodiment, the minimum medium contains a reduced amount of source of sulfur, preferably a reduced amount of MgSO₄ and/or (NH₄)₂SO₄. In particular, the minimum medium may not contain more than 1 g/l of MgSO₄ or may contain approximately 1 g/l of MgSO₄. It may also not contain more than 0.2 g/l of (NH₄)₂SO₄ or may contain approximately 0.2 g/l of (NH₄)₂SO₄.

According to another embodiment, the minimum medium is deprived of one or more vitamins of the B group. In particular, the minimum medium may not contain choline and/or inositol. Preferably, it contains neither choline nor inositol.

The organoleptic qualities of the flour compositions can be evaluated by means of a tasting composition prepared by mixing 5-10% of composition of flour of microalgae, 0.5-2% of sugar, 0.1-0.5% of vanilla flavoring and skimmed milk, the percentages being expressed by weight of the tasting composition, the composition being homogenized before heating it at 60-85° C. for 2-10 minutes.

The microalgae can be selected from Chlorella protothecoides, Chlorella kessleri, Chlorella minutissima, Chlorella sp., Chlorella sorokiniama, Chlorella luteoviridis, Chlorella vulgaris, Chlorella reisiglii, Chlorella ellipsoidea, Chlorella saccarophila, Parachlorella kessleri, Parachlorella beijerinkii, Prototheca stagnora and Prototheca moriformis.

Preferably, the microalgae belong to the genus Chlorella and very particularly preferably are Chlorella protothecoides microalgae.

According to one embodiment, the microalgae are deprived of chlorophyll pigments. These microalgae can in particular be cultured in darkness or are incapable of producing or have a reduced ability to produce chlorophyll pigments.

The present invention also relates to a composition of flour of lipid-rich microalgae which is obtained by the process according to the invention and to the use of this flour composition in the preparation of a food composition.

### DETAILED DESCRIPTION OF THE INVENTION

Within the meaning of the invention, a composition of flour of microalgae exhibits an “optimized sensory profile” or an “optimized organoleptic quality” when its evaluation by a sensory panel in food formulation (for example in an ice cream or in a tasting composition as described below) concludes that off-notes which detrimentally affect the organoleptic quality of said food formulations containing these compositions of flour of microalgae are absent.

The term “organoleptic quality” is understood to mean the property of a food in terms of taste, odor, appearance, color and consistency.

These off-notes are associated with the presence of specific undesirable odorous and/or aromatic molecules which are characterized by a perception threshold corresponding to the minimum value of the sensory stimulus necessary for the triggering of a sensation.

The “optimized sensory profile” or “optimized organoleptic quality” is then rendered by a sensory panel by the achievement of the best scores on a scale of evaluation of the four sensory criteria (appearance, texture, savors and flavors).

The term “total content” is understood to mean the sum of the contents for each of the volatile organic compounds of the list.

As used here, the term “approximately” refers to a value+/−20%, 10%, 5% or 2%.

Within the meaning of the present invention, the term “flour of microalgae” should be understood in its broadest interpretation and as denoting, for example, a composition comprising a plurality of particles of biomass of microalgae. The biomass of microalgae is derived from cells of microalgae, which can be whole or ruptured, or a mixture of whole and ruptured cells.

A number of documents of the state of the art, such as the international patent application WO 2010/120923, describe methods for the preparation and use in food of the biomass of Chlorella microalgae.

The microalgae with which the present invention is concerned are thus preferably microalgae of the genus Chlorella, more particularly Chlorella protothecoides, more particularly still Chlorella microalgae deprived of chlorophyll pigments, by any method known per se to a person skilled in the art (either in that the culturing is carried out in darkness or because the strain has been mutated so as to no longer produce these pigments). In particular, the lipid-rich microalgae can be chosen, nonexhaustively, from Chlorella protothecoides, Chlorella kessleri, Chlorella minutissima, Chlorella sp., Chlorella sorokiniama, Chlorella luteoviridis, Chlorella vulgaris, Chlorella reisiglii, Chlorella ellipsoidea, Chlorella saccarophila, Parachlorella kessleri, Parachlorella beijerinkii, Prototheca stagnora and Prototheca moriformis. Thus, in a very specific embodiment, the composition of flour of microalgae is a composition of Chlorella flour and in particular of Chlorella protothecoides flour.

The fermentative process described in the patent application WO 2010/120923 makes possible the production of a number of compositions of flour of microalgae of variable organoleptic quality.

The culture medium recommended in this patent application is a complex fermentation medium comprising:
a source of carbon (glucose, fructose, sucrose, galactose, xylose, mannose, rhamnose, arabinose, and the like),
a source of nitrogen (such as proteins, soybean flour, yeast extract, water from steeping of maize, and the like),
elements in the form of traces (zinc, boron, cobalt, copper, manganese and molybdenum, introduced in the form of ZnCl₂, H₃BO₄, CoCl₂.6H₂O, CuCl₂.2H₂O, MnCl₂.4H₂O and (NH₄)₆Mo₇O₂₄.4H₂O),
optionally a pH buffer and
phosphate (phosphate salts can be used as source of phosphorus).

For the strains kept alive by the University of Texas at Austin for its collection of microalgae (UTEX), various media are provided (cf. their internet site http://www.utew.org/).

Furthermore, in the patent application WO 2010/120923, it is specified that a high content of oil can be produced by providing a source of carbon in excess and while limiting nitrogen.

However, nowhere in this document is the use of a minimum medium mentioned or suggested, whether to bring about the growth of the microalga or to promote the production of flour compositions exhibiting an optimized sensory profile.

A minimum medium is conventionally defined as a medium comprising only the chemical elements strictly necessary for the growth of the microalga, in a form which can be used by the microalgae having no specific requirement.

The minimum medium then contains:
a source of carbon and energy: generally glucose;
a source of potassium and phosphorus: for example K₂HPO₄;
a source of nitrogen and sulfur: for example (NH₄)₂SO₄;
a source of magnesium and sulfur: for example MgSO₄. 7H₂O;
a source of calcium: for example CaCl₂.2H₂O;
a source of iron: for example FeSO₄.7H₂O;
sources of trace elements: salts of Cu, Zn, Co, B, Mn, Mo; and
vitamins (thiamine, biotin, vitamin B12, and the like).

In point of fact, as will be exemplified below, the applicant company has found that, if the sulfur supply of this minimum medium is further reduced and/or certain vitamins (in particular choline and inositol, which belong to vitamins of the B group) are removed, compositions of flour of these microalgae are then obtained which exhibit an optimized sensory profile.

In order to determine this sensory profile, the applicant company has defined a very simple tasting matrix which nevertheless makes it possible to carry out an organoleptic evaluation similar to that obtained with much more complex and very different recipes, such as an ice cream or a brioche.

This tasting matrix for compositions of flour of microalgae comprises:
5-10% of composition of flour of microalgae, preferably approximately 7%;
0.5-2% of sugar, preferably approximately 1%;
0.1-0.5% of vanilla flavoring, preferably approximately 0.25%; and
the remainder as skimmed milk, preferably approximately 91.75%;

the percentages being expressed by weight of the tasting composition.

The preparation of a tasting composition as described above is homogenized, heated at 60-85° C., preferably approximately 75° C., for 2-10 minutes, preferably approximately 5 minutes.

A sensory panel is defined in order to test and evaluate the organoleptic qualities of a composition of flour of microalgae included in the tasting composition as described above.

This sensory panel is formed in order to evaluate the sensory properties of different batches of compositions of flour of microalgae, in particular of flour of Chlorella protothecoides biomass.

A group of people, at least 10, 20 or 30, in particular approximately 15, is gathered together in order to evaluate the descriptors of several compositions of flour of microalgae, preferably in comparison with a reference sample of flour of microalgae which is identified as conforming, that is to say of acceptable organoleptic quality, (reference batch No. 1) and another sample of highly unacceptable organoleptic quality (reference batch No. 2).

Preferably, the compositions of flour of microalgae are tested in the form of a tasting composition according to the present document. Alternatively, the compositions can be tested in any other form desired by a person skilled in the art, for example in the form of an ice cream or of a breadmaking product, such as a brioche.

Preferably, the reference products as presented in the following table are associated with each descriptor:

| | |
|---|---|
| Descriptors | Reference |

| | | |
|---|---|---|
| Appearance | Color (from light | |
| | to dark) | |
| Texture | Topping | Whole milk + 5% cream |
| Savors | Sweet | 1% Sucrose |
| Flavors | Mushroom | 100 g of mushrooms in |
| | | 100 ml of cold water/x4 |
| | | dilution |
| | Cereals | 10% Ebly solution |
| | Butter/dairy | |
| | product | |
| | Rancid oil | 1.5% Oxidized oil |
| | Vegetable | Highly unacceptable |
| | aftertaste | composition of flour of |
| | | microalgae |

Of course, a person skilled in the art can define other reference products if he so desires.

At each tasting session, several products, for example four to five, are evaluated with regard to each descriptor in comparison with a reference batch or sample, preferably a reference batch or sample regarded as of acceptable organoleptic quality.

All the products are evaluated one after the other, on scales ranging, for example, from 1 to 9, in the following way:
Value of 1: the descriptor evaluated is not present in the product;
Value of 5: the descriptor evaluated is present in the product exactly in the same way as in the reference product of acceptable organoleptic quality;
Value of 9: the descriptor evaluated is very present in the product.

The reference batch of acceptable organoleptic quality is a composition of flour of microalgae which conforms in the sense that it exhibits the “satisfactory” sensory profile of all these descriptors. The reference batch of highly unacceptable organoleptic quality is a batch which does not satisfy the descriptors relating to the aromatic notes, that is to say to the descriptors Savors and Flavors, as it exhibits, for example, a significant vegetable aftertaste.

It is important to note that the reference batch of acceptable organoleptic quality is not necessarily the composition of flour of microalgae exhibiting the optimum sensory profile: it is preferably a composition of flour of microalgae perceived by the sensory panel as “satisfactory”, in particular exhibiting a grade of 5, over all the descriptors tested. In this embodiment, the compositions of flour of microalgae which are tested are categorized by the sensory panel on either side of this reference batch of acceptable organoleptic quality.

Generally, the compositions tested are categorized by the sensory panel with respect to the reference batch(es) of acceptable or unacceptable organoleptic quality, preferably with respect to the reference batch(es) of acceptable organoleptic quality.

Thus, the first stage results in the classification, as a function of their organoleptic quality, of the various compositions of flour of microalgae which are tested.

In particular, analyses of variance (ANOVA) are carried out in order to evaluate the discriminating capacity of the descriptors (descriptors of which the p-value associated with the Fisher test—type-3 ANOVA—is less than 0.20 for the Composition effect in the model descriptor˜Composition+judge). The Composition effect is interpreted as the discriminating capacity of the descriptors: if there is no effect (Critical Probability >0.20), the compositions were not discriminated according to this criterion.

The smaller the critical probability, the more discriminating the descriptor. A Principal Component Analysis (PCA) is subsequently carried out in order to obtain sensory mapping of the compositions and a simultaneous representation of all the compositions over all the descriptors.

Thus, the present invention relates to a process for the preparation, by the fermentative route, of compositions of flour of lipid-rich microalgae of optimized organoleptic quality.

In the process according to the invention, the microalgae, preferably microalgae of the genus Chlorella and more particularly Chlorella protothecoides microalgae, are cultured in a minimum medium. The biomass thus obtained can subsequently be converted into flour of microalgae.

The minimum medium as used in the present invention comprises a source of carbon, potassium, phosphorus, nitrogen, magnesium, calcium, iron, trace elements and vitamins.

According to a specific embodiment, the minimum medium comprises the following compounds: glucose, KH₂PO₄, NaH₂PO₄, MgSO₄, (NH₄)₂SO₄, CaCl₂, FeSO₄, MnSO₄, CoSO₄, CuSO₄, ZnSO₄, H₃BO₃, Na₂MoO₄, thiamine, biotin, vitamin B12, calcium pantothenate and p-aminobenzoic acid, and optionally inositol and/or choline chloride.

Preferably, this minimum medium comprises a reduced amount of source of sulfur and/or is deprived of one or more vitamins of the B group.

According to one embodiment, the minimum medium comprises a reduced amount of source of sulfur.

As used here, the term “reduced amount” means, preferably, that the amount present in the medium is less than the amount known for meeting the requirements of the strain. Preferably, this term means an amount lower by 20, 25, 30, 35, 40, 45 or 50% than the amount known for meeting the requirements of the strain. Alternatively, a reduced amount can mean that the amount present in the medium only 50, 55, 60, 65, 70, 75 or 80% of the requirements of the strain cultured.

The sulfur present in the minimum medium originates essentially from the macroelements containing this element. This is because the contribution of the microelements is normally negligible. Thus, according to one embodiment, the sulfur present in the minimum medium originates essentially from MgSO₄ and/or (NH₄)₂SO₄.

Thus, according to a specific embodiment, the minimum medium used comprises at most the equivalent of approximately 1 g/l of MgSO₄ and of approximately 0.2 g/l of (NH₄)₂SO₄ as source of sulfur. Preferably, the minimum medium used comprises at most the equivalent of 1 g/l of MgSO₄ and of 0.2 g/l of (NH₄)₂SO₄ as source of sulfur.

According to a preferred embodiment, the minimum medium used comprises the equivalent of approximately 1 g/l of MgSO₄ and of approximately 0.2 g/l of (NH₄)₂SO₄ as source of sulfur and more preferably the equivalent of 1 g/l of MgSO₄ and of 0.2 g/l of (NH₄)₂SO₄.

According to one embodiment, the minimum medium is deprived of one or more vitamins of the B group.

Preferably, the minimum medium does not comprise choline and/or inositol. More particularly preferably, the minimum medium comprises neither choline nor inositol.

Preferably, the microalgae are cultured in the minimum medium throughout the duration of the fermentation, that is to say until sufficient biomass is obtained to be converted into flour. Typically, the fermentation has a duration of more than 50 hours, preferably between 50 and 150 hours. The biomass produced is preferably greater than 100 or 150 g/l of medium and very particularly preferably between 100 and 250 g/l of medium.

As used here, the term “lipid-rich microalgae” more particularly denotes microalgae producing a biomass comprising more than 30, 35, 40 or 45% of lipids by dry weight of biomass.

The microalgae are preferably deprived of chlorophyll pigments. In particular, the fermentation can be carried out in darkness. Alternatively, the microalgae can also be incapable of producing or have a reduced ability to produce chlorophyll pigments.

Thus, according to a preferred embodiment, the microalgae are cultured under heterotrophic conditions, that is to say without light, using a carbon-based substrate (preferably glucose) as source of carbon and energy.

According to a preferred embodiment, the biomass obtained contains few or no organoleptically undesirable compounds, such as the products of oxidative decomposition of monounsaturated fatty acids.

In particular, the biomass contains few or no products of oxidative decomposition of oleic acid. Preferably, the content of linoleic acid (product of oxidative decomposition of oleic acid) is less than 18, 15, 10, 8 or 7% by weight, with respect to the total weight of the fatty acids in the dry biomass.

Although it can be used on a smaller scale, the process is preferably carried out on the industrial scale, that is to say on medium-capacity (of approximately 1 to 100 m³) and high-capacity (of more than 100 m³) fermenters. According to one embodiment, the process is carried out on fermenters with a capacity of at least 1, 10, 25, 50, 75, 100, 500 or 1000 m³.

The process according to the invention can also comprise a stage of conversion of the biomass obtained into flour of microalgae. This stage can involve any method known to a person skilled in the art for obtaining flours of microalgae.

The present invention also relates to a composition of flour of lipid-rich microalgae obtained according to the process of the present invention.

It also relates to the use of this flour composition for the preparation of food compositions, such as foodstuffs, nutritional supplements, confectionery or drinks.

A better understanding of the invention will be obtained with the help of the following examples, which are meant to be illustrative and nonlimiting.

### EXAMPLES

### Example 1: Production of Lipid-Rich Chlorella Protothecoides Microalgae—Reduction of the Contribution of MgSO₄ or of Vitamins

The strain used is Chlorella protothecoides UTEX 250.

### Preculture:

500 ml of medium in a 2 l Erlenmeyer flask;
Composition of the medium (in g/l or mg/l):

| | | |
|---|---|---|
| Macroelements | Glucose | 40 |
| (g/l) | K₂HPO₄ | 3 |
| | Na₂HPO₄ | 3 |
| | MgSO₄•7H₂O | 0.25 |
| | (NH₄)₂SO₄ | 1 |
| | Citric acid | 1 |
| | Clerol FBA 3107 | 0.1 |
| | (defoamer) | |
| Microelements | CaCl₂•2H₂O | 30 |
| and vitamins | FeSO₄•7H₂O | 1 |
| (mg/l) | MnSO₄•1H₂O | 8 |
| | CoSO₄•7H₂O | 0.1 |
| | CuSO₄•5H₂O | 0.2 |
| | ZnSO₄•7H₂O | 0.5 |
| | H₃BO₃ | 0.1 |
| | Na₂MoO₄•2H₂O | 0.4 |
| | Thiamine•HCl | 1 |
| | Biotin | 0.015 |
| | B12 | 0.01 |
| | Calcium pantothenate | 0.03 |
| | p-Aminobenzoic acid | 0.06 |

Incubation takes place under the following conditions: duration: 72 h; temperature: 28° C.; stirring: 110 rpm (Infors Multitron incubator).

The preculture is subsequently transferred into a 30 l fermenter of Sartorius type.

### Culture for Producing Biomass:

The base medium is as follows:

| | | Base medium | | |
|---|---|---|---|---|
| | | (Test 1) | Test 2 | Test 3 |
| Macro- | Glucose | 40 | 40 | 40 |
| elements | KH₂PO₄ | 0.9 | 0.9 | 0.9 |
| (g/l) | NaH₂PO₄ | 0.7 | 0.7 | 0.7 |
| | MgSO₄•7H₂O | 1.7 | 1 | 1.7 |
| | (NH₄)₂SO₄ | 0.2 | 0.2 | 0.2 |
| | Clerol FBA 3107 | 0.3 | 0.3 | 0.3 |
| | (defoamer) | | | |
| Micro- | CaCl₂•2H₂O | 20 | 20 | 20 |
| elements | FeSO₄•7H₂O | 6 | 6 | 6 |
| and | MnSO₄•1H₂O | 20 | 20 | 20 |
| vitamins | CoSO₄•7H₂O | 0.05 | 0.05 | 0.05 |
| (mg/l) | CuSO₄•5H₂O | 0.3 | 0.3 | 0.3 |
| | ZnSO₄•7H₂O | 25 | 25 | 25 |
| | H₃BO₃ | 7 | 7 | 7 |
| | Na₂MoO₄•2H₂O | 1 | 1 | 1 |
| | Inositol | 100 | 100 | 0 |
| | Choline chloride | 100 | 100 | 0 |
| | Thiamine•HCl | 3 | 3 | 3 |
| | Biotin | 0.05 | 0.05 | 0.05 |
| | B12 | 0.03 | 0.03 | 0.03 |
| | Calcium | 0.1 | 0.1 | 0.1 |
| | pantothenate | | | |
| | p-Aminobenzoic | 0.1 | 0.1 | 0.1 |
| | acid | | | |

The contribution of MgSO₄ is reduced to 1 g/l in test 2 and the choline chloride and the inositol are removed in test 3.

The initial volume (Vi) of the fermenter is adjusted to 7 l after inoculation. It is brought to 15-20 l in the end.

The parameters for carrying out the fermentation are as follows:

| | |
|---|---|
| Temperature | 28° C. |
| pH | 6.8 with 28% w/w NH₃ and then 5N KOH |
| pO₂ | >20% (maintained by stirring) |
| Stirring | 300 rpm mini |
| Air flow rate | 15 l/min |

When the residual concentration of glucose falls below 10 g/l, contribution of glucose in the form of a 700 g/l concentrated solution is carried out continuously, so as to maintain the content of glucose between 0 and 20 g/l in the fermenter.

When 1000 g of glucose have been consumed and when the biomass has reached a concentration of 70 g/l, the aqueous ammonia is replaced with potassium hydroxide for the regulation of pH. This makes it possible for the biomass to accumulate lipids.

### Results:

| | | | | | Grade of |
|---|---|---|---|---|---|
| | | | | | the |
| | | Duration | Biomass | | sensory |
| Test | Medium | (h) | (g/l) | % Lipids | panel |
| 1 | Base | 95 | 180 | 50 | 7 |
| 2 | Base with MgSO₄ | 94 | 178 | 48 | 4 |
| | reduced to 1 g/l | | | | |
| 3 | Base without | 96 | 181 | 49 | 4 |
| | inositol or choline | | | | |

The higher the grade given by the sensory panel, the more the composition is characterized by the presence of off-notes.

The flour compositions obtained with tests 2 and 3 obtain a grade of less than 5, that is to say a lower grade than the reference composition of satisfactory organoleptic quality. The compositions of tests 2 and 3 thus have better organoleptic properties than those of the reference composition.

These results thus show that the limitation of the medium in sulfur, in particular by reducing the concentration of MgSO₄, which is the main source of sulfur, or the absence of certain vitamins, in particular inositol and/or choline, results in a significant reduction in the level of the off-notes and thus in an improvement in the organoleptic quality of the composition.

1-23. (canceled)

24. A process for the preparation of compositions of flour of microalgae of optimized organoleptic quality comprising culturing the microalgae in a minimum culture medium under fermentative conditions, wherein the minimum culture medium comprises chemically defined elements comprising a source of carbon, a source of potassium, a source of phosphorus, a source of nitrogen, a source of sulfur, a source of magnesium, a source of calcium, a source of iron, sources of trace elements, and one or more vitamins;
wherein:
(a) the minimum culture medium does not comprise choline and inositol;
(b) the minimum culture medium comprises greater than zero, but not more than about 1 g/l of MgSO₄;
(c) the minimum culture medium comprises greater than zero, but not more than about 0.2 g/l of (NH₄)₂SO₄;
wherein the microalgae in the minimum culture medium reaches a cell density of at least about 70 g/L; and
wherein the microalgae belong to the genus Chlorella.

25. The process as claimed in claim 24, wherein the minimum culture medium contains about 1 g/l of MgSO₄.

26. The process as claimed in claim 24, wherein the minimum culture medium contains about 0.2 g/l of (NH₄)₂SO₄.

27. The process as claimed in claim 24, wherein the organoleptic qualities of the flour are evaluated using a tasting composition prepared by mixing 5-10% of the composition of flour of microalgae, 0.5-2% of sugar, 0.1-0.5% of vanilla flavoring and skimmed milk, the percentages being expressed by weight of the tasting composition, the tasting composition being homogenized before heating it at 60-85° C. for 2-10 minutes.

28. The process as claimed in claim 24, wherein the microalgae are selected from the group consisting of Chlorella protothecoides, Chlorella kessleri, Chlorella minutissima, Chlorella sp., Chlorella sorokiniama, Chlorella luteoviridis, Chlorella vulgaris, Chlorella reisiglii, Chlorella ellipsoidea, and Chlorella saccarophila.

29. The process as claimed in claim 24, wherein the microalgae is Chlorella protothecoides.

30. The process as claimed in claim 24, wherein the microalgae are cultured in darkness.

31. The process as claimed in claim 24, wherein the microalgae are incapable of producing or have a reduced ability to produce chlorophyll pigments.

32. The process as claimed in claim 24, wherein the minimum medium comprises the following compounds: glucose, KH₂PO₄, NaH₂PO₄, MgSO₄, (NH₄)₂SO₄, CaCl₂, FeSO₄, MnSO₄, CoSO₄, CuSO₄, ZnSO₄, H₃BO₃, Na₂MoO₄, thiamine, biotin, vitamin B12, calcium pantothenate and p-aminobenzoic acid.

33. The process as claimed in claim 24, wherein the microalgae obtained by the method contains less than about 18% by weight of linoleic acid, with respect to the total weight of the fatty acids in the dry microalgae.

34. The process as claimed in claim 24, wherein the microalgae obtained by the method contains less than about 15% by weight of linoleic acid, with respect to the total weight of the fatty acids in the dry microalgae.

35. The process as claimed in claim 24, wherein the microalgae obtained by the method contains less than about 10% by weight of linoleic acid, with respect to the total weight of the fatty acids in the dry microalgae.

36. The process as claimed in claim 24, wherein the microalgae obtained by the method contains less than about 8% by weight of linoleic acid, with respect to the total weight of the fatty acids in the dry microalgae.

37. The process as claimed in claim 24, wherein the microalgae obtained by the method contains less than about 7% by weight of linoleic acid, with respect to the total weight of the fatty acids in the dry microalgae.
